(19) United States
(12) Patent Application Publication
(10) Pub. No.: US 2025/0255680 A1
(43) Pub. Date: Aug. 14, 2025

(54) SURGICAL ROBOTIC SYSTEM AND METHOD FOR PREDICTING CABLE WEAR AND FAILURE

(71) Applicant: Covidien LP, Mansfield, MA (US)

(72) Inventors: Richard L. Croft, Mead, CO (US); Michael B. Lyons, Boulder, CO (US); David J. Van Tol, Boulder, CO (US); Matthew S. Cowley, Frederick, CO (US)

(21) Appl. No.: 19/040,568

(22) Filed: Jan. 29, 2025

Related U.S. Application Data
(60) Provisional application No. 63/551,209, filed on Feb. 8, 2024.

(51) Int. Cl. A61B 34/30; A61B 34/00

(52) U.S. Cl. CPC A61B 34/30; A61B 34/71

(57) ABSTRACT

A surgical robotic system includes an instrument, at least one sensor, a processor, and a memory. The instrument includes an end effector and a plurality of cables. The at least one sensor detects a vibration signature produced by the plurality of cables. The memory is coupled to the processor and has instructions stored thereon which, when executed by the processor, cause the surgical robotic system to: store an vibration signal threshold corresponding to an vibration signature of the plurality of cables when the plurality of cables are in a first state; detect, by the at least one sensor, an in-use a vibration signature produced by the plurality of cables during use of the instrument in surgery; determine, based on the in-use vibration signature, an in-use vibration signal corresponding to the plurality of cables during use of the instrument; determine, based on a comparison of the stored vibration signal threshold and the in-use vibration signal, an in-use state of the plurality of cables; and output an alert indicating a condition of the plurality of cables corresponding to the in-use state of the plurality of cables when the in-use vibration signal exceeds the vibration signal threshold.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body.

Surgical robotic instruments are actuated by one or more cables, which may snap during use due to high strain placed on them during actuation. Cable snapping may result in unintentional, uncontrolled movement of portions of the instruments, e.g., the end effector.

### SUMMARY

According to one aspect of the present disclosure, a surgical robotic system is described. The surgical robotic system includes an instrument, at least one sensor, a processor, and a memory. The instrument includes an end effector and a plurality of cables. The at least one sensor is configured to detect a vibration signature produced by the plurality of cables. The memory stores a vibration signal threshold corresponding to a vibration signature of the plurality of cables prior to use of the plurality of cables in the instrument in surgery, and is coupled to the processor and has instructions stored thereon which, when executed by the processor, cause the surgical robotic system to: store a vibration signal corresponding to a vibration signature of the plurality of cables when the plurality of cables are in a first state; detect, by the at least one sensor, an in-use vibration signature produced by the plurality of cables during use of the instrument in surgery; determine, based on the in-use vibration signature, an in-use vibration signal corresponding to the plurality of cables during use of the instrument; and determine, based on a comparison of the stored vibration signal threshold and the in-use vibration signal, an in-use state of the plurality of cables; and output an alert indicating a condition of the plurality of cables corresponding to the in-use state of the plurality of cables when the in-use vibration signal exceeds the vibration signal threshold.

In another aspect of the present disclosure, the in-use state of the plurality of cables may correspond to a failure state of at least one cable of the plurality of cables.

In yet another aspect of the present disclosure, the surgical robotic system may be configured to receive a user command for controlling the instrument, and the instructions, when executed by the processor, may further cause the surgical robotic system to disable execution of the user command when the in-use state of the plurality of cables corresponds to the failure state of at least one cable of the plurality of cables.

In a further aspect of the present disclosure, the surgical robotic system may further include a surgeon console including a display screen for displaying a graphical user interface. The graphical user interface may be configured to receive a user input for setting a trigger event.

In yet a further aspect of the present disclosure, the instructions, when executed by the processor, may further cause the surgical robotic system to determine, based on the trigger event and the in-use vibration signal, a third state of the plurality of cables.

In another aspect of the present disclosure, the third state may correspond to a failure state of a filar of at least one cable of the plurality of cables.

In yet another aspect of the present disclosure, the surgical robotic system may further include an instrument drive unit coupled to the instrument. The instrument drive unit may include a plurality of motors and a controller. The instructions, when executed by the processor, may further cause the surgical robotic system to control the plurality of motors to reduce tension in the plurality of cables.

In a further aspect of the present disclosure, the stored and in-use vibration signals may correspond to a frequency of the vibration signature of the plurality of cables prior to and during use of the instrument.

In another aspect of the present disclosure, the alert may include at least one of a visual or an audio indication that at least one cable of the plurality of cables has reached a predetermined level of wear.

According to one aspect of the present disclosure, a processor-implemented method of cable failure detection is disclosed. The method includes: storing a vibration signal threshold corresponding to a vibration signature of a plurality of cables of a robotic surgical instrument when the plurality of cables are in a first state; detecting, by at least one sensor, an in-use vibration signature produced by the plurality of cables during use of the instrument in surgery, wherein the at least one sensor is configured to detect a vibration signature produced by the plurality of cables; determining, based on the in-use vibration signature, an in-use vibration signal corresponding to the plurality of cables during use of the instrument in surgery; and determining, based on a comparison of the stored vibration signal threshold and the in-use vibration signal, a second state of the plurality of cables; and outputting an alert indicating a condition of the plurality of cables corresponding to an in-use state of the plurality of cables when the in-use vibration signal exceeds the vibration signal threshold.

In another aspect of the present disclosure, the in-use state of the plurality of cables may correspond to a failure state of at least one cable of the plurality of cables.

In yet another aspect of the present disclosure, the method may further include disabling entry of a user input for controlling the instrument when the in-use state of the plurality of cables corresponds to the failure state of at least one cable of the plurality of cables.

In a further aspect of the present disclosure, the method may further include displaying a graphical user interface on a display screen of a surgeon console, the graphical user interface configured to receive a user input for setting a trigger event.

In yet a further aspect of the present disclosure, the method may further include determining, based on the trigger event and the in-use vibration signal, a third state of the plurality of cables.

In another aspect of the present disclosure, the third state may correspond to a failure state of a filar of at least one cable of the plurality of cables.

In yet another aspect of the present disclosure, the method may further include controlling, by an instrument drive unit coupled to the instrument, the plurality of cables to reduce tension in the plurality of cables.

In a further aspect of the present disclosure, the stored and in-use vibration signals may correspond to a frequency of the vibration signature of the plurality of cables prior to and during use.

In yet a further aspect of the present disclosure, the alert may include at least one of a visual or an audio indication that at least one cable of the plurality of cables has reached a predetermined level of wear.

According to one aspect of the present disclosure, one or more non-transitory processor-readable media are disclosed. The media stores instructions which, when executed by one or more processors, cause performance of: storing a vibration signal threshold corresponding to a vibration signature of a plurality of cables of a robotic surgical instrument when the plurality of cables are in a first state; detecting, by at least one sensor, an in-use vibration signature produced by the plurality of cables during use of the instrument in surgery, wherein the at least one sensor is configured to detect a vibration signature produced by the plurality of cables; determining, based on the in-use vibration signature, an in-use vibration signal corresponding to the plurality of cables during use of the instrument in surgery; and determining, based on a comparison of the stored vibration signal threshold and the in-use vibration signal, a second state of the plurality of cables; and outputting an alert indicating a condition of the plurality of cables corresponding to an in-use state of the plurality of cables when the in-use vibration signal exceeds the vibration signal threshold.

### BRIEF DESCRIPTION OF THE DRAWINGS

Various embodiments of the present disclosure are described herein with reference to the drawings wherein:

FIG. 1 is a perspective view of a surgical robotic system including a control tower, a console, and one or more surgical robotic arms each disposed on a movable cart according to an aspect of the present disclosure;

FIG. 2 is a perspective view of a surgical robotic arm of the surgical robotic system of FIG. 1 according to an aspect of the present disclosure;

FIG. 3 is a perspective view of a movable cart having a setup arm with the surgical robotic arm of the surgical robotic system of FIG. 1 according to an aspect of the present disclosure;

FIG. 4 is a schematic diagram of a computer architecture of the surgical robotic system of FIG. 1 according to an aspect of the present disclosure;

FIG. 5 is a plan schematic view of movable carts of FIG. 1 positioned about a surgical table according to an aspect of the present disclosure;

FIG. 6 is a perspective view, with parts separated, of an instrument drive unit and a surgical instrument according to an aspect of the present disclosure;

FIG. 7 is a side, cross-sectional view of the instrument according to an aspect of the present disclosure, for use in the surgical robotic system of FIG. 1;

FIG. 8 is a top, perspective view of the end effector of the instrument of FIG. 7, according to an aspect of the present disclosure;

FIG. 9 shows the end effector in various configurations according to aspects of the present disclosure;

FIG. 10 is a perspective view of a system for predicting cable wear and breakage according to an aspect of the present disclosure; and

FIG. 11 is a flow chart of a method for predicting cable wear and breakage according to an aspect of the present disclosure.

### DETAILED DESCRIPTION

Embodiments of the presently disclosed surgical robotic system are described in detail with reference to the drawings, in which like reference numerals designate identical or corresponding elements in each of the several views.

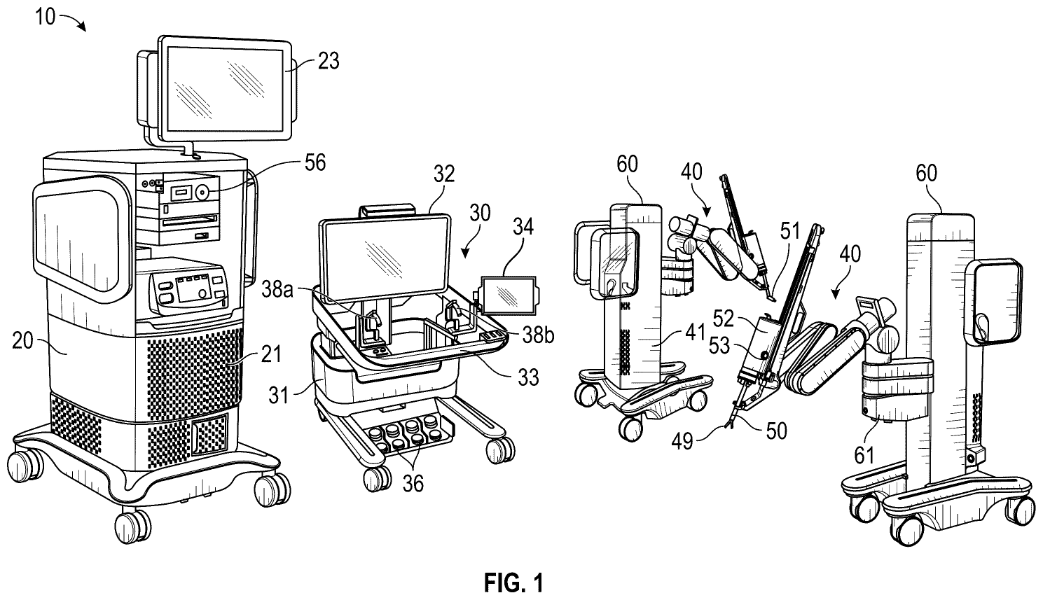

With reference to FIG. 1, a surgical robotic system 10 includes a control tower 20, which is communicatively coupled to all of the components of the surgical robotic system 10 including a surgeon console 30 and one or more movable carts 60. Each of the movable carts 60 includes a robotic arm 40 having an instrument 50 coupled thereto. The robotic arms 40 also couple to the movable carts 60. The surgical robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured to apply a surgical clip onto tissue. However, it will be understood that various types of surgical instruments for use during minimally invasive surgical procedures are contemplated and within the scope of this disclosure.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first screen 32, which displays a video feed of the surgical site provided by camera 51 disposed on the robotic arm 40, and a second screen 34, which displays a user interface for controlling the surgical robotic system 10. The first screen 32 and second screen 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a screen 23, which may be a touchscreen that may display the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted by using any logic processor (e.g., control circuit) adapted to execute algorithms, calculations, and/or set of instructions described herein.

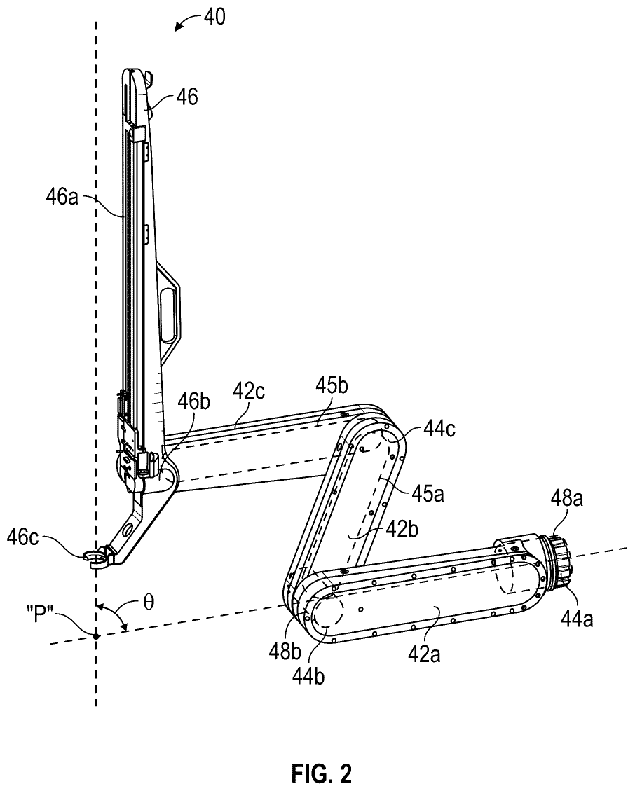

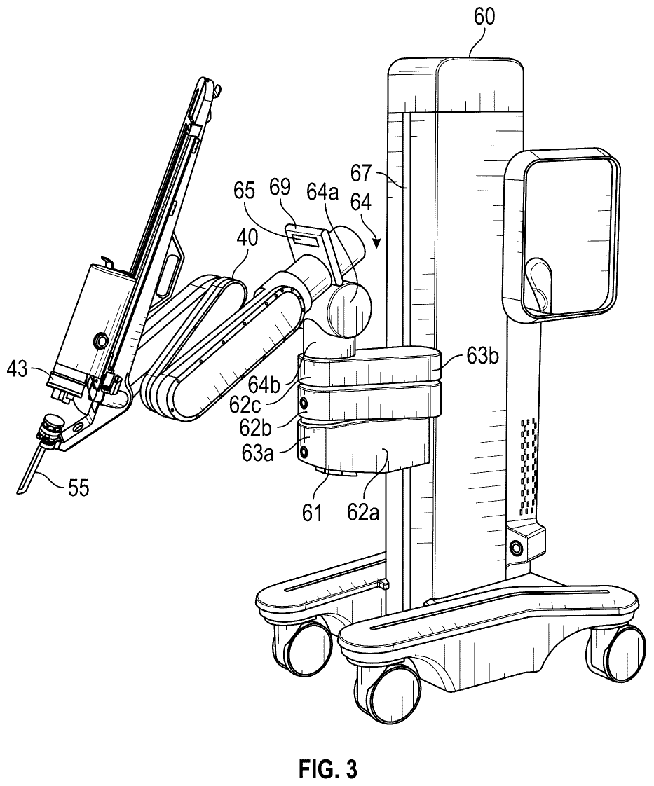

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c arc interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle 0. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components of an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52.

The robotic arm 40 also includes a plurality of manual override buttons 53 (FIG. 1) disposed on the IDU 52 and the setup arm 61, which may be used in a manual mode. The user may press one or more of the buttons 53 to move the component associated with the one or more buttons 53.

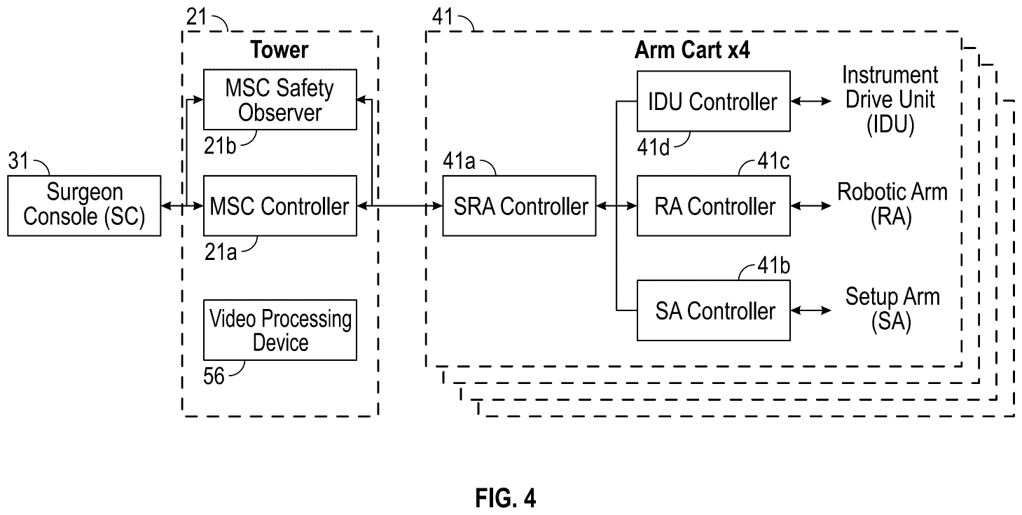

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c. In aspects, handle controller 38a may be substituted for and/or employed in conjunction with handle controller 38b.

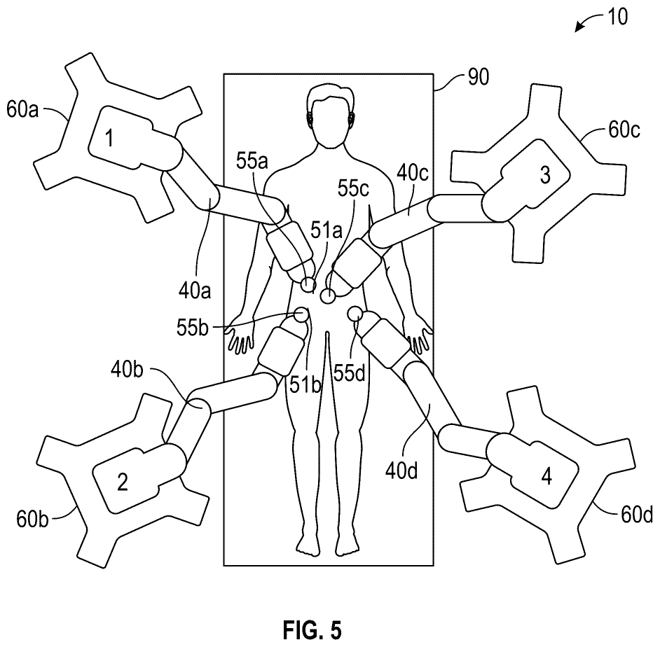

With reference to FIG. 5, the surgical robotic system 10 is set up around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

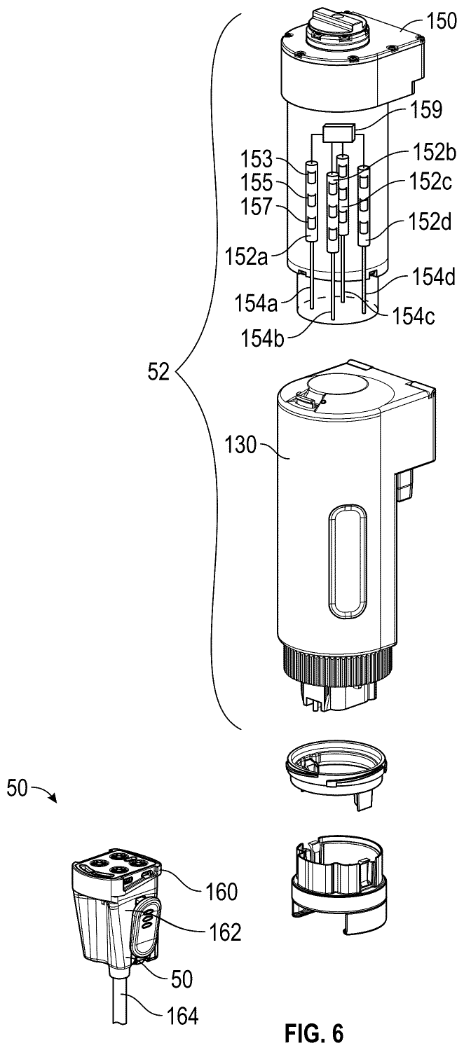

With reference to FIG. 6, the IDU 52 is shown in more detail and is configured to transfer power and actuation forces from its motors 152a-d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

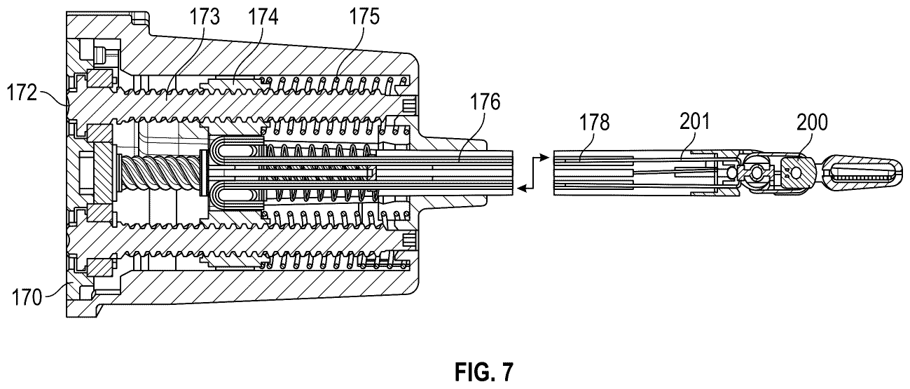

The IDU 52 includes a motor pack 150 and a sterile barrier housing 130. Motor pack 150 includes motors 152a-d for controlling various operations of the instrument 50. The instrument 50 is removably couplable to IDU 52. As the motors 152a-d of the motor pack 150 are actuated, rotation of the drive transfer shafts 154a, 154b, 154c, 154d of the motors 152a-d, respectively, is transferred to the drive assemblies of the instrument 50. The instrument 50 is configured to transfer rotational forces/movement supplied by the IDU 52 (e.g., via the motors 152a-d of the motor pack 150) into longitudinal movement or translation of the cables or drive shafts to effect various functions of an end effector 200 (FIG. 7).

Each of the motors 152a-d includes a current sensor 153, a torque sensor 155, and a position sensor 157. For conciseness only operation of the motor 152a is described below, however, it will be understood that motors 152b-d may operate in a similar manner. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by motor 152a. Position sensor 157 may be any device that provides a sensor signal indicative of the number of rotations of the motor 152a, such as a mechanical encoder or an optical encoder. Parameters which are measured and/or determined by position sensor 157 may include speed, distance, revolutions per minute, position, and the like. The sensor signals from sensors 153, 155, 157 are transmitted to the IDU controller 41d, which then controls the motors 152a-d based on the sensor signals. In particular, the motors 152a-d are controlled by an actuator controller 159, which controls torque outputted and angular velocity of the motors 152a-d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

With reference to FIGS. 6 and 7, instrument 50 includes an adapter 160 having a housing 162 at a proximal end portion thereof and an elongated shaft 164 that extends distally from housing 162. Housing 162 of instrument 50 is configured to selectively couple to IDU 52 to enable motors 152a-d of IDU 52 to operate the end effector 200 of the instrument 50. Housing 162 of instrument 50 supports a drive assembly 170 that mechanically and/or electrically cooperates with motors 152a-d of IDU 52. Drive assembly 170 of instrument 50 includes a plurality of rotatable input couplers 172, each of which is actuated by the corresponding motors 152a-d. The couplers 172 are threaded and include one corresponding drive nut 174 threadably coupled to a corresponding drive screw 173. The drive nuts 174 are prevented from rotational movement by the housing of the drive assembly 170. The drive nuts 174 move longitudinally along the couplers 172 as the couplers 172 are rotated by the motors 152a-d and are maintained in the longitudinal position via tension springs 175. Each of the drive nuts 174 is coupled to a corresponding cable 201a-d via a drive rod 176 and a crimp 178. Thus, as drive nuts 174a-d are moved longitudinally, the cables 201a-d are also moved longitudinally, thereby tensioning the cables 201a-d.

The surgical instrument also includes an end effector 200 coupled to the elongated shaft 164. The end effector 200 may include any number of degrees of freedom allowing the end effector 200 to articulate, pivot, etc., relative to the elongated shaft 164. The end effector 200 may be any suitable surgical end effector configured to treat tissue, such as a dissector, grasper, sealer, stapler, etc.

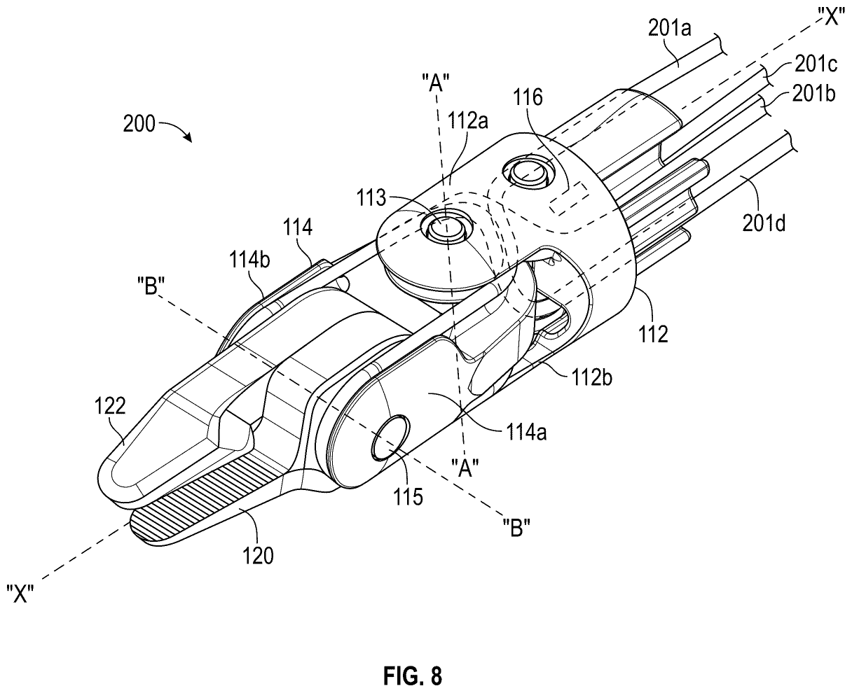

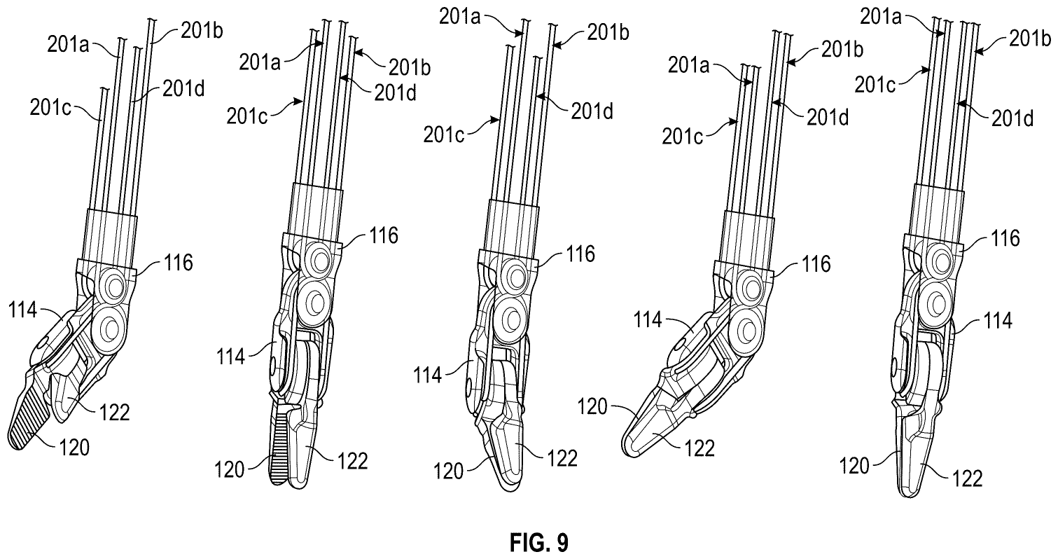

As shown in FIGS. 7, 8, and 9, the end effector 200 may include a pair of opposing jaws 120 and 122 that are movable relative to each other. The jaws 120 and 122 may be grippers as shown or any other suitable type of jaws, e.g., shears, sealers, etc. In embodiments, the end effector 200 may include a proximal portion 112 having a first pin 113 and a distal portion 114. The end effector 200 may be actuated using a plurality of cables 201a-d routed through proximal and distal portions 112 and 114 around their respective pulleys 112a, 112b, 114a, 114b, which are integrally formed as arms of the proximal and distal portions 112 and 114. Each of the cables 201a-d is actuated by a respective motor 152a-d via corresponding couplers disposed in adapter 160. In embodiments, the end effector 200, namely, the distal portion 114 and the jaws 120 and 122, may be articulated about the axis “A-A” to control a yaw angle of the end effector with respect to a longitudinal axis “X-X”. The distal portion 114 includes a second pin 115 with a pair of jaws including a first jaw 120 and a second jaw 122 pivotably coupled to the second pin 115. The jaws 120 and 122 are configured to pivot about an axis “B-B” defined by the second pin 115 allowing for controlling a pitch angle of the jaws 120 and 122 as well as opening and closing the jaws 120 and 122. The yaw, pitch, and jaw angles between the jaws 120 and 122 as they are moved between open and closed positions are controlled by adjusting the tension and/or length and direction (e.g., proximal or distal) of the cables 201a-d as shown in FIG. 8. The end effector 200 also includes a cable displacement sensor 116 configured to measure position of the cables 201. Thus, the end effector 200 may have three degrees of freedom, yaw, pitch, and jaw angle between jaws 120 and 122. Examples of control algorithms for a cable actuated instrument 50 are described in International Patent Application No. PCT/US2022/019703, “Surgical Robotic System for Realignment of Wristed Instruments,” filed on Mar. 10, 2022.

After long periods of usage of the instrument 50, the plurality of cables 201a-d may become worn. In particular, after extended usage of end effectors such as shears (e.g., monopolar curved shears), repetitive shear and wrist actuation may cause twisted filars (or fibers) within the plurality of cables 201a-d to fray and ultimately snap. After the plurality of cables 201a-d have become significantly worn, or after continued usage of the instrument 50 having cables 201a-d which are significantly worn, cable failure (e.g., snap or breakage) may occur, which requires replacement of instrument 50 and a ceasing of operations within the surgical robotic system 10. Moreover, once cable breakage occurs, the end effector 200 is driven to an approximate right-angle position relative to the elongated shaft 164 due to tension applied by other (i.e., unsnapped) cables, which are still loaded under a tension force. Thus, it may be difficult or impossible to remove the instrument 50, having an articulated end effector 200, through the access port 55. As a result, the clinician may enact an instrument slack procedure to reduce tension on the other (unsnapped cables). Examples of instrument slack procedures for the plurality of cables 201a-d are described further below.

Therefore, it is important for a clinician, or a surgical robotic system, to predict breakage of the plurality of cables 201a-d prior to a total cable failure. The present disclosure provides a system and a method configured to predict and/or detect wearing of the plurality of cables, 201a-d, such as high side cables 201b and 201c (i.e., cables which are under relatively greater tension loads), using sounds or vibration signatures emitted from cables 201a-d that are detected by vibration signature sensors.

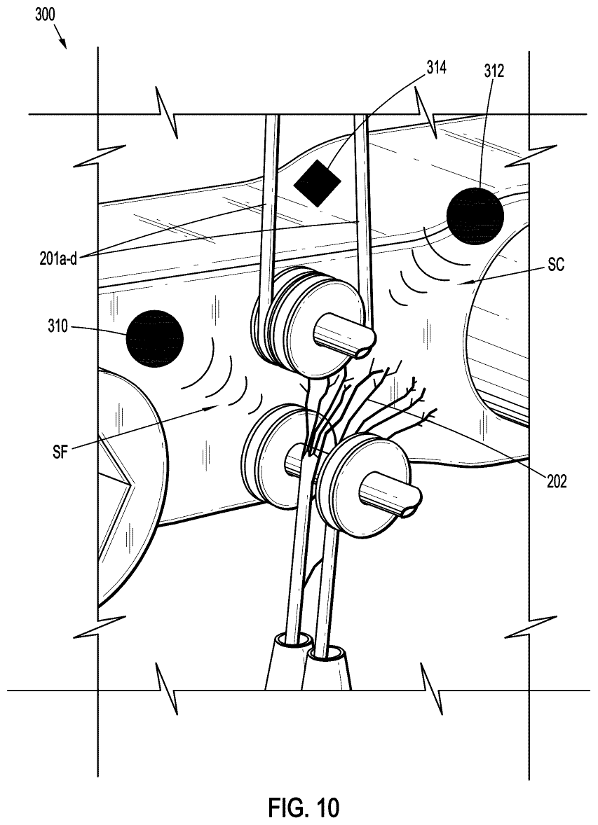

With reference to FIG. 10, a system 300 for predicting cable wear and failure is shown, which forms a part of the surgical robotic system 10. System 300 includes a plurality of vibration signature sensors 310, 312 configured to measure a frequency of a vibration signature produced by cables 201a-d and/or individual filars 202 of the cables 201a-d. The vibration signature sensors 310, 312 may include a recording device 314 for capturing the vibration signature, such as a microphone. In embodiments, the vibration signature sensors 310, 312 may be placed proximal to cables 201a-d, although alternative placements throughout the surgical robotic system 10 are contemplated and within the scope of this disclosure. It will be understood that while two sensors 310, 312 are shown next to cables 102a-d and further described herein, any number and/or arrangement of sensors 310, 312 may be included in system 300. For example, the system 300 may function with only one vibration signature sensor 310 or 312.

The vibration signature sensors 310, 312 may be configured to detect a characteristic sound or signature, e.g., a vibration signature emitted from cables 201a-d and/or individual filars 202 due to vibration and/or breakage thereof. The vibration signature may be the result of vibration through the air, vibration of the cables and filars, and/or another part of the surgical robotic instrument 10. For example, vibration signature sensors 310, 312 may detect soundwaves emitted within a predetermined distance from the components of the system 300. In another example, vibration signature sensors 310, 312 may be configured to detect vibration of the components of the system 300, such as vibrations detected from being physically positioned on said components. As used herein, a vibration signature may include a signal within the normal range of hearing, an ultrasonic frequency (e.g., over 20 kHz), a specific cable and/or filar vibration pattern, sound waves, electrical continuity, electrical resistance, an audio signal, and/or another signal and/or signature known by one skilled in the art, as discussed further below.

The vibration signature may be converted to a vibration signal for processing by the surgical robotic system 10. The system may use the resulting signal for comparison with another signal. In aspects, the system may compare the signal to a vibration signal threshold, as further discussed below. This vibration signal threshold may be preprogrammed into surgical robotic system 10, and/or set as a system default upon installation.

For example, the vibration signature sensors 310, 312 may be configured to record a vibration signature “SF” produced by the individual filars 202. The vibration signature “SF” may indicate snapping and/or vibration of individual filars 202 as cables 201a-d wear under tension (e.g., from shear and wrist actuation of the end effector 200 of the instrument 50). In particular, the vibration signature sensors 310, 312 may be programmed to detect a specific frequency of the vibration signature “SF” of the filars 202 when snapping, which corresponds to breakage. If the frequency of the vibration signature “SF” matches a preprogrammed frequency or threshold (e.g., a vibration signal threshold) corresponding to filar breakage, the vibration signature sensors 310, 312 may transmit an alert to the surgical robotic system 10. For example, the vibration signature sensors 310, 312 may emit an audible or visual alert signal or cause the surgical robotic system 10 to emit an audible or visual alert signal. The alert signal may be used to alert the clinician that a filar breakage has occurred. Therefore, the clinician and/or the surgical robotic system 100 may be able to act appropriately.

In another example, the vibration signature sensors 310, 312 may be configured to record a vibration signature “SC” produced by the cables 201a-d. A frequency of the vibration signature “SC” may change over time as the individual filars 202 of the cables 201a-d snap, which may indicate a health state of the plurality of cables 201a-d. In particular, the vibration signature sensors 310, 312 may be configured to detect specific changes in the frequency of the vibration signature “SC”, which correspond to a likelihood of cable failure for the cables 201a-d. In doing so, the vibration signature sensors 310, 312 continuously monitor the vibration signature “SC”, which is analyzed in comparison to a baseline vibration signature “SC” obtained from a vibration signal threshold prior to usage. If the vibration signature “SC” of the cables 201a-d exceeds a frequency threshold in relation to a baseline value, the vibration signature sensors 310, 312 may transmit an alert to the surgical robotic system 10. For example, the vibration signature sensors 310, 312 may emit an audible or visual alert signal or cause the surgical robotic system 10 to emit an audible or visual alert signal. The alert signal may be used to alert the clinician that the cables 201a-d are likely to fail. Therefore, the clinician and/or the surgical robotic system 10 may be able cease operation and/or inspect and/or replace the instrument 50 and/or cables 201a-d. In aspects, the vibration signature sensors 310, 312 may be configured to record a vibration signature “SC” of an individual cable 201a-d. This solves the problem of determining which cable 201a-d broke and may be useful for analyzing whether a particular cable or group of cables is experiencing more failure than others.

Alternatively, in embodiments, the surgical robotic system 10 may be able to adjust a tension of the cables 201a-d and/or a torque of the motors 152a-d, in order to extend a use life of the instrument 50 and prevent immediate failure. For example, a tension force on the cables 201a-d may be released or reduced by a certain factor for certain cables 201a-d, which have been observed by vibration signature sensors 310, 312 to have filars 202 within those the cables 201a-d that exhibit signs of fraying, which may thus prevent a snapping of the filars 202 altogether and which may extend the use life of the instrument 50. In another example, a torque produced by motors 152a-d may be reduced, thereby allowing the clinician to move an instrument 50 with reduced force. The adjustments may be made intraoperatively, thereby allowing the clinician to continue using the instrument 50 without interruption for a period of time.

In embodiments, the vibration signature sensors 310, 312 may include an accelerometer to measure specific vibration or acoustical signals and/or vibration or acoustical lengths of the cables 201a-d and/or the filars 202. For example, if a vibration speed of one or more of the cables 201a-d exceeds a preprogrammed threshold, the vibration signature sensors 310, 312 may transmit an audible or visual alert signal and/or cause the surgical robotic system 10 to emit an audible or visual alert signal, which will similarly alert a clinician that snapping of filars 202 has occurred and/or potential failure of cables 201a-d may soon occur. In doing so, the vibration signature sensors 310, 312 may provide an alternative means of detecting a frequency of a vibration signal, such as vibration signals “SF”, “SC”, for the filars 202 and cables 201a-d, respectively. For example, the accelerometer(s) may be able to detect an inaudible and/or an ultrasonic frequency band emitted by the cables 201a-d and/or the filars 202, which is incapable of detection by a microphone. In another example, the vibration signature sensors 310, 312, may be configured to measure changes in tension per unit length of cables 210a-d. For example, as more filars 202 break, a tension of the cables 201a-d may increase. In doing so, the vibration signature sensors 310, 312 may be able to measure a breakage percentage of filars 202 to determine the remaining use life of the cables 201a-d.

In embodiments, the signature sensors 310, 312 may be replaced by, or additionally configured to detect electrical continuity and/or resistance through the cables 201a-d. For example, such electrical continuity sensors may detect a change in resistance through cables 201a-d indicating a partial cable failure, or an open circuit indicating a total cable failure.

Alternatively, in embodiments, the surgical robotic system 10 may induce a vibration in one of more cables 102a-d and/or individual filars 202 in order to detect a level of tension on the cables indicative of a health state. The vibration may be induced through tapping, plucking, or an alternative means of initiating vibration. Generally, the vibration may be induced without movement and/or prior to movement caused by use of the surgical robotic system 10.

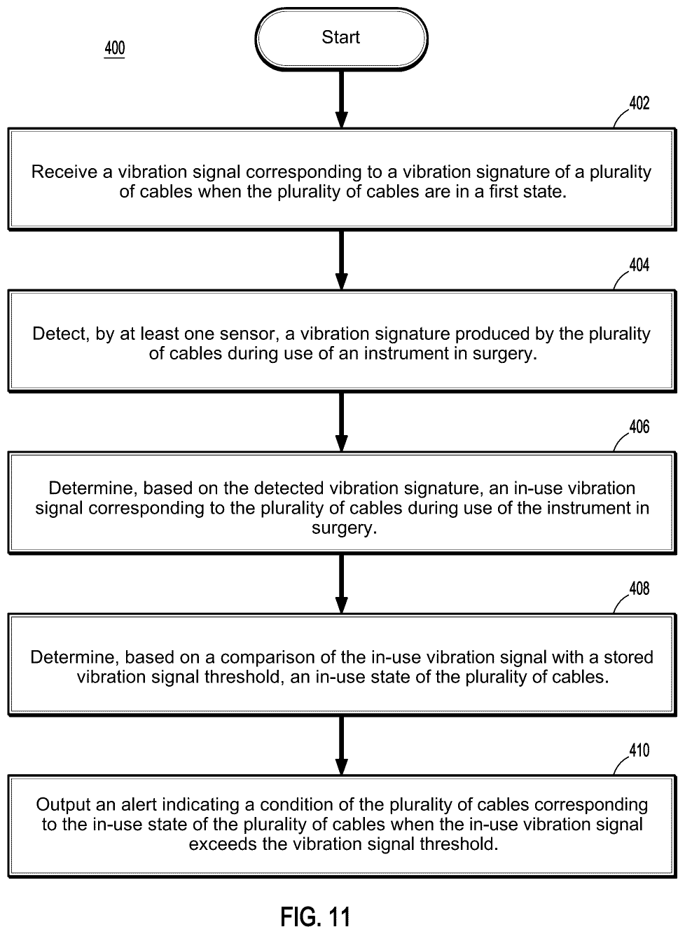

With reference to FIG. 11, a method 400 for predicting and/or detecting cable wear and failure is implemented by the vibration signature sensors 310, 312, as software instructions executable by any suitable processor of the surgical robotic system 10, such as the IDU controller 41d, the main controller 21a, etc., among others contemplated and within the scope of this disclosure.

Initially, at step 402, the processor receives a vibration signal corresponding to a vibration signature of the plurality of cables 201a-d and/or filars 202 which has been recorded when the plurality of cables 201a-d are in a first state, typically when the instrument 50 is first constructed. This signal may be a digitized version of a vibration signature obtained from the surgical robotic system 10. In addition, a vibration signal threshold is stored in the surgical robotic system 10, which may be used as a threshold for comparison with a vibration signal, as discussed below. For example, during manufacturing and/or installation of the instrument 50, vibration sensors may be used to record baseline vibration signature measurements related to cables 201a-d and/or filars 202, which will correspond to the vibration signal threshold. The vibration sensors may record a baseline vibration signature “SF” of filars 202. This baseline vibration signature “SF” may correspond to the initial frequency of a vibration produced by filars 202 before use of instrument 50 in surgery. In another example, audio sensors may record a baseline vibration signature “SC” of the cables 201a-d. This baseline vibration signature “SC” of the cables 201a-d may correspond to an initial frequency of a vibration produced by cables 201a-d before use of instrument 50.

At step 404, the processor causes the vibration signature sensors 310, 312 to record vibration signature measurements related to cables 201a-d and/or filars 202 during use of instrument 50 in surgery. For example, the vibration signature sensors 310, 312 may record a vibration signature “SF” of filars 202. This vibration signature “SF” corresponds to the frequency of a vibration produced by filars 202 during use of instrument 50. In addition and/or alternatively, the processor may determine a specific frequency of the vibration signature “SF” of the filars 202 that will correspond to filar breakage. This specific frequency of vibration signal “SF” of the filars 202 will be set as a trigger event.

In embodiments, the baseline vibration signature “SF”, “SC”, for the filars 202 and cables 201a-d, respectively, may be automatically transmitted to or provided to the processor of the surgical robotic system 10 based on detection of a specific instrument 50, such as monopolar curved shears (e.g., a vibration signal threshold). In addition and/or alternatively, the processor may determine a specific frequency of vibration signal “SC” of the cables 201a-d that will correspond to cable wear and/or breakage. This specific frequency of vibration signal “SC” of the cables 201a-d will be set as a trigger event. Once obtained, all baseline vibration signal measurements and/or specific frequencies will be converted to vibration signals for cable wear and failure prediction analysis.

In embodiments, the controller 21a also outputs a prompt on one of the GUIs of the screens 23, 32, 34. The prompt requests input from the clinician related to the procedure, including the types of instrument(s) 50 used. For example, the clinician may select a procedure that requires the use of monopolar curved shears. In response to the selection, baseline vibration signature measurements related to cables 201a-d and/or filars 202 may be preprogrammed (e.g., a vibration signal threshold). For example, a baseline vibration signature “SF” of the filars 202 and/or a baseline vibration signature “SC” of the cables 201a-d may be set. In addition and/or alternatively, a specific frequency corresponding to breakage of filars 202 of cables 201a-d in monopolar curved shears may be set. Further, the preprogrammed baseline vibration signature measurements may also be manually input by the clinician.

Next, at step 406, the recorded vibration signature measurements are used to predict, determine and/or detect cable wear and failure. For example, the vibration signature sensors 310, 312 will continuously measure the live frequency of vibration signals “SC” produced by cables 201a-d and the vibration signatures “SF” produced by individual filars 202 during use of the instrument 50 (e.g., the monopolar curved shears). These frequencies of live vibration signatures “SC”, “SF”, for the filars 202 and cables 201a-d, respectively, will be compared, in step 408, to the stored vibration signature threshold(s), i.e., the baseline vibration signature measurements received by the processor in step 402. In doing so, cable wear and/or failure may be detected by comparing, in step 408, the measured frequencies of live vibration signals “SC”, “SF”', for the filars 202 and cables 201a-d, respectively, to the corresponding thresholds that are indicative of the filar breakage and/or cable failure. For example, the vibration signature sensors 310, 312 may detect that a frequency of vibration signature “SF”, for the filars 202 has surpassed a specific threshold in relation to the baseline frequency of vibration signature “SF” for the filars 202, indicating that a filar breakage is likely to occur. In addition and/or alternatively, the vibration signature sensors 310, 312 may detect a frequency of vibration signature “SF” for the filars 202 corresponding to the programmed trigger event, such as a high-pitched snapping noise indicating breakage of a filar 202.

In another example, the vibration signature sensors 310, 312 may detect that a frequency of vibration signature “SC” for the cables 201a-d has surpassed a specific vibration signal threshold in relation to the frequency of vibration signature “SC” for the cables 201a-d, indicating that a cable failure is likely to occur. In addition and/or alternatively, the vibration signature sensors 310, 312 may detect a frequency of vibration signature “SC” for the cables 201a-d corresponding to the programmed trigger event or a wear sound. In aspects, the vibration signature sensors 310, 312 may detect sounds of the cables 102a-d, pulleys 112a, 112b, 114a, 114b, and/or an axle system changing beyond their normal vibrational signature range. Sounds may include a galling sound, hum, etc. indicating future failure of cables 201a-d.

At step 410, once cable wear and/or failure is detected, the controller 21a may transmit a signal to the surgical robotic system 10 to perform one or more actions. For example, the controller 21a may cause the surgical robotic system 10 to emit an audible alert indicating cable failure. In addition and/or alternatively, the controller 21a may disable further user input commands for controlling the instrument 50 and output a prompt on one of the GUIs of the screens 23, 32, 34. The prompt may be an alert indicating that (1) a cable failure is predicted; or (2) a cable failure has occurred. In doing so, the user will stop attempting to control the instrument 50, preventing further damage. The prompt may include an indication of the location of the cable 102a-d that is worn and/or has failed, thereby instructing an operator which cable 102a-d requires repair, may soon require repair, or a remaining useful life for the cables 102a-d if operated under normal load conditions. In aspects, a tension of cables 201a-d and/or a torque of the motors 152a-d may be reduced. This may occur automatically or through user intervention.

In another case, such as where a cable failure is predicted, the clinician may be presented with multiple options. First, the clinician may select a “continue” option to ignore the cable failure alert, which will cause controller 21a to reenable further user input commands for controlling the instrument 50 to continue the procedure. Second, the clinician may select a “reduce tension” or “reduce torque” option to adjust a tension of cables 201a-d or a torque produced by motors 152a-d. For example, the user may lower a tension of cables 201a-d or a torque of the motors 152a-d by 50%, releasing a specific amount of tension on filars 202 to extend the lifespan of the instrument 50. Third, the clinician may select a “replace instrument” option to replace the instrument 50.

In another case, such as where a cable failure is detected, the clinician may be required to cease all operations and replace instrument 50 in order to re-enable further user input commands for controlling the instrument 50. Therefore, the clinician cannot continue operating until a new instrument 50 including new cables 201a-d is installed.

Following selection of the “replace instrument” option or the detection of cable failure, at step 408, the system enters an instrument exchange process during which the instrument 50 is withdrawn from the access port 55. During the instrument exchange process and prior to withdrawal of the instrument 50, all of the motors 152a-d are driven so that all of the input couplers 172 are driven to remove tension in the cables 201a-d, or at least the input couplers 172 of the cables 201a-d that are not broken are driven in this manner. Thus, the IDU controller 41d determines which cable 201a-d is broken and corresponding motor 152a-d to only control the motor(s) coupled to unbroken cables.

The IDU 52 may verify that sufficient slack has been achieved based on torque or position. The couplers 172a-d may be driven until the measured torque is minimal or approximately “0” with the drive nuts 174a-d being driven distally to remove tension. Conversely, the couplers 172a-d may be driven to a desired position with the drive nuts 174a-d being driven distally to remove tension. Once the remaining cables 201a-d are slackened, the end effector 200 may be passively manipulated.

Once the instrument 50 has been replaced, the controller 21a will reenable further user input commands for controlling the instrument 50 and will output a prompt on one of the GUIs of the screens 23, 32, 34. The output will include a health state of the new instrument 50 and options for additional user input, such as specific triggering events and baseline vibration signature settings. For example, at any time, a clinician may select a “health state” option on one of the GUIs of the screens 23, 32, 34 to induce a vibration in one or more cables 102a-d and/or individual filars 202. The surgical robotic system 10 may analyze the vibration and output a detected level of tension on the cables 102a-d and/or filars 202. Thus, the steps 402-410 may repeat iteratively throughout a procedure until the clinician selects a “complete” button on one of the GUIs of the screens 23, 32, 34 indicating that the procedure is complete.

It should be understood that various aspects disclosed herein may be combined in different combinations than the combinations specifically presented in the description and accompanying drawings.

While the present disclosure specifically shows and describes systems and methods for monitoring conditions of the use-life of cables of instruments, and predicting and/or monitoring for failure of such cables, it is understood and within the scope of the present disclosure that the systems and methods described herein may be applied towards collisions of instruments (i.e., unintentional collisions with other instruments or hard objects), towards the monitoring of the useful life and/or the predicting of failure of various other components of the instruments (e.g., drive screws, coupling nuts, and the like), and/or towards the monitoring of disconnection or separation of the cables from crimps or coupling features which connect the cables to respective drive rods or the like (e.g., J-hook crimps, etc.).

Further aspects and embodiments of the present disclosure are set out in the below numbered clauses:
1. A surgical robotic system comprising:
   an instrument including an end effector and a plurality of cables;
   at least one sensor configured to detect a vibration signature produced by the plurality of cables;
   a processor; and
   a memory coupled to the processor, the memory storing a vibration signal threshold corresponding to a vibration signature of the plurality of cables prior to use of the plurality of cables in the instrument in surgery and having instructions stored thereon which, when executed by the processor, cause the surgical robotic system to:
      receive a vibration signal corresponding to a vibration signature of the plurality of cables when the plurality of cables are in a first state;
      detect, by the at least one sensor, a vibration signature produced by the plurality of cables during use of the instrument in surgery;
      determine, based on the detected vibration signature, an in-use vibration signal corresponding to the plurality of cables during use of the instrument in surgery;
      determine, based on a comparison of the in-use vibration signal with the stored vibration signal threshold, an in-use state of the plurality of cables; and
      output an alert indicating a condition of the plurality of cables corresponding to the in-use state of the plurality of cables when the in-use vibration signal exceeds the vibration signal threshold.
2. The surgical robotic system of clause 1, wherein the in-use state of the plurality of cables corresponds to a failure state of at least one cable of the plurality of cables.
3. The surgical robotic system of clause 2, wherein the instrument is configured to receive a user command for controlling the instrument, and wherein the instructions, when executed by the processor, further cause the surgical robotic system to:
   disable execution of the user command when the in-use state of the plurality of cables corresponds to the failure state of at least one cable of the plurality of cables.
4. The surgical robotic system of clause 1, further comprising a surgeon console including a display screen for displaying a graphical user interface, the graphical user interface configured to receive a user input for setting a trigger event.
5. The surgical robotic system of clause 4, wherein the instructions, when executed by the processor, further cause the surgical robotic system to:
   determine, based on the trigger event and the in-use vibration signal, a third state of the plurality of cables.
6. The surgical robotic system of clause 5, wherein the third state corresponds to a failure state of a filar of at least one cable of the plurality of cables.
7. The surgical robotic system of clause 1, further comprising an instrument drive unit coupled to the instrument, the instrument drive unit including a plurality of motors and a controller, and wherein the instructions, when executed by the processor, further cause the surgical robotic system to:
   control the plurality of motors to reduce tension in the plurality of cables.
8. The surgical robotic system of clause 1, wherein the in-use vibration signature produced by the plurality of cables includes sounds of cable galling.
9. The surgical robotic system of clause 8, wherein the in-use and stored vibration signals correspond to a frequency of the vibration signatures of the plurality of cables prior to and during use of the surgical instrument.
10. The surgical robotic system of clause 1, wherein the alert includes at least one of a visual or an audio indication that at least one cable of the plurality of cables has reached a predetermined level of wear.
11. A processor-implemented method of cable failure detection, the method comprising:
   storing a vibration signal threshold corresponding to a vibration signature of a plurality of cables of a robotic surgical instrument when the plurality of cables are in a first state;
   detecting, by at least one sensor, an in-use vibration signature produced by the plurality of cables during use of the instrument in surgery, wherein the at least one sensor is configured to detect a vibration signature produced by the plurality of cables;
   determining, based on the in-use vibration signature, an in-use vibration signal corresponding to the plurality of cables during use of the instrument in surgery;
   determining, based on a comparison of the stored vibration signal threshold and the in-use vibration signal, a second state of the plurality of cables; and
   outputting an alert indicating a condition of the plurality of cables corresponding to an in-use state of the plurality of cables when the in-use vibration signal exceeds the vibration signal threshold.
12. The processor-implemented method of clause 11, wherein the in-use state of the plurality of cables corresponds to a failure state of at least one cable of the plurality of cables.
13. The processor-implemented method of clause 12, further comprising:
   disabling entry of a user command for controlling the instrument when the in-use state of the plurality of cables corresponds to the failure state of at least one cable of the plurality of cables.
14. The processor-implemented method of clause 11, further comprising:
   displaying a graphical user interface on a display screen of a surgeon console, the graphical user interface configured to receive a user input for setting a trigger event.
15. The processor-implemented method of clause 14, further comprising:
   determining, based on the trigger event and the in-use vibration signal, a third state of the plurality of cables.
16. The processor-implemented method of clause 15, wherein the third state corresponds to a failure state of a filar of at least one cable of the plurality of cables.
17. The processor-implemented method of clause 11, further comprising:
   controlling, by an instrument drive unit coupled to the instrument, the plurality of cables to reduce tension in the plurality of cables.
18. The processor-implemented method of clause 11, wherein the stored and in-use vibration signals correspond to a frequency of the vibration signatures of the plurality of cables prior to and during use of the surgical instrument.
19. The processor-implemented method of clause 11, wherein the alert includes at least one of a visual or an audio indication that at least one cable of the plurality of cables has reached a predetermined level of wear.
20. One or more non-transitory processor-readable media storing instructions which, when executed by one or more processors, cause performance of:
   storing a vibration signal threshold corresponding to a vibration signature of a plurality of cables of a robotic surgical instrument when the plurality of cables are in a first state;
   detecting, by at least one sensor, an in-use vibration signature produced by the plurality of cables during use of the instrument in surgery, wherein the at least one sensor is configured to detect a vibration signature produced by the plurality of cables;
   determining, based on the in-use vibration signature, an in-use vibration signal corresponding to the plurality of cables during use of the instrument in surgery;
   determining, based on a comparison of the stored vibration signal threshold and the in-use vibration signal, an in-use state of the plurality of cables; and
   outputting an alert indicating a condition of the plurality of cables corresponding to the in-use state of the plurality of cables when the in-use vibration signal exceeds the vibration signal threshold.

It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, the above-described servers and computing devices.

While the description above refers to particular aspects of the present disclosure, it will be understood that many modifications may be made without departing from the spirit thereof. Additional steps and changes to the order of the algorithms can be made while still performing the key teachings of the present disclosure. Thus, the accompanying claims are intended to cover such modifications as would fall within the true scope and spirit of the present disclosure. The presently disclosed aspects are, therefore, to be considered in all respects as illustrative and not restrictive, the scope of the disclosure being indicated by the appended claims rather than the foregoing description. Unless the context indicates otherwise, any aspect disclosed herein may be combined with any other aspect or aspects disclosed herein. All changes that come within the meaning of, and range of, equivalency of the claims are intended to be embraced therein.

What is claimed is:

1. A surgical robotic system comprising:
an instrument including an end effector and a plurality of cables;
at least one sensor configured to detect a vibration signature produced by the plurality of cables;
a processor; and
a memory coupled to the processor, the memory storing a vibration signal threshold corresponding to a vibration signature of the plurality of cables prior to use of the plurality of cables in the instrument in surgery and having instructions stored thereon which, when executed by the processor, cause the surgical robotic system to:
receive a vibration signal corresponding to a vibration signature of the plurality of cables when the plurality of cables are in a first state;
detect, by the at least one sensor, a vibration signature produced by the plurality of cables during use of the instrument in surgery;
determine, based on the detected vibration signature, an in-use vibration signal corresponding to the plurality of cables during use of the instrument in surgery;
determine, based on a comparison of the in-use vibration signal with the stored vibration signal threshold, an in-use state of the plurality of cables; and
output an alert indicating a condition of the plurality of cables corresponding to the in-use state of the plurality of cables when the in-use vibration signal exceeds the vibration signal threshold.

2. The surgical robotic system of claim 1, wherein the in-use state of the plurality of cables corresponds to a failure state of at least one cable of the plurality of cables.

3. The surgical robotic system of claim 2, wherein the instrument is configured to receive a user command for controlling the instrument, and wherein the instructions, when executed by the processor, further cause the surgical robotic system to:
disable execution of the user command when the in-use state of the plurality of cables corresponds to the failure state of at least one cable of the plurality of cables.

4. The surgical robotic system of claim 1, further comprising a surgeon console including a display screen for displaying a graphical user interface, the graphical user interface configured to receive a user input for setting a trigger event.

5. The surgical robotic system of claim 4, wherein the instructions, when executed by the processor, further cause the surgical robotic system to:
determine, based on the trigger event and the in-use vibration signal, a third state of the plurality of cables.

6. The surgical robotic system of claim 5, wherein the third state corresponds to a failure state of a filar of at least one cable of the plurality of cables.

7. The surgical robotic system of claim 1, further comprising an instrument drive unit coupled to the instrument, the instrument drive unit including a plurality of motors and a controller, and wherein the instructions, when executed by the processor, further cause the surgical robotic system to:
control the plurality of motors to reduce tension in the plurality of cables.

8. The surgical robotic system of claim 1, wherein the in-use vibration signature produced by the plurality of cables includes sounds of cable galling.

9. The surgical robotic system of claim 8, wherein the in-use and stored vibration signals correspond to a frequency of the vibration signatures of the plurality of cables prior to and during use of the surgical instrument.

10. The surgical robotic system of claim 1, wherein the alert includes at least one of a visual or an audio indication that at least one cable of the plurality of cables has reached a predetermined level of wear.

11. A processor-implemented method of cable failure detection, the method comprising:
storing a vibration signal threshold corresponding to a vibration signature of a plurality of cables of a robotic surgical instrument when the plurality of cables are in a first state;
detecting, by at least one sensor, an in-use vibration signature produced by the plurality of cables during use of the instrument in surgery, wherein the at least one sensor is configured to detect a vibration signature produced by the plurality of cables;
determining, based on the in-use vibration signature, an in-use vibration signal corresponding to the plurality of cables during use of the instrument in surgery;
determining, based on a comparison of the stored vibration signal threshold and the in-use vibration signal, a second state of the plurality of cables; and
outputting an alert indicating a condition of the plurality of cables corresponding to an in-use state of the plurality of cables when the in-use vibration signal exceeds the vibration signal threshold.

12. The processor-implemented method of claim 11, wherein the in-use state of the plurality of cables corresponds to a failure state of at least one cable of the plurality of cables.

13. The processor-implemented method of claim 12, further comprising:
disabling entry of a user command for controlling the instrument when the in-use state of the plurality of cables corresponds to the failure state of at least one cable of the plurality of cables.

14. The processor-implemented method of claim 11, further comprising:
displaying a graphical user interface on a display screen of a surgeon console, the graphical user interface configured to receive a user input for setting a trigger event.

15. The processor-implemented method of claim 14, further comprising:
determining, based on the trigger event and the in-use vibration signal, a third state of the plurality of cables.

16. The processor-implemented method of claim 15, wherein the third state corresponds to a failure state of a filar of at least one cable of the plurality of cables.

17. The processor-implemented method of claim 11, further comprising:
controlling, by an instrument drive unit coupled to the instrument, the plurality of cables to reduce tension in the plurality of cables.

18. The processor-implemented method of claim 11, wherein the stored and in-use vibration signals correspond to a frequency of the vibration signatures of the plurality of cables prior to and during use of the surgical instrument.

19. The processor-implemented method of claim 11, wherein the alert includes at least one of a visual or an audio indication that at least one cable of the plurality of cables has reached a predetermined level of wear.

20. One or more non-transitory processor-readable media storing instructions which, when executed by one or more processors, cause performance of:
storing a vibration signal threshold corresponding to a vibration signature of a plurality of cables of a robotic surgical instrument when the plurality of cables are in a first state;
detecting, by at least one sensor, an in-use vibration signature produced by the plurality of cables during use of the instrument in surgery, wherein the at least one sensor is configured to detect a vibration signature produced by the plurality of cables;
determining, based on the in-use vibration signature, an in-use vibration signal corresponding to the plurality of cables during use of the instrument in surgery;
determining, based on a comparison of the stored vibration signal threshold and the in-use vibration signal, an in-use state of the plurality of cables; and
outputting an alert indicating a condition of the plurality of cables corresponding to the in-use state of the plurality of cables when the in-use vibration signal exceeds the vibration signal threshold.
